(19) United States
(12) Patent Application Publication
(10) Pub. No.: US 2017/0173228 A1
(43) Pub. Date: Jun. 22, 2017

(54) DEVICE FOR CLEANING A MEDICAL VACUUM PUMP

(71) Applicant: MEDELA HOLDING AG, Baar (CH)

(72) Inventor: Hilmar Ehlert, Hergiswil (CH)

(21) Appl. No.: 15/129,756

(22) PCT Filed: Mar. 31, 2015
(86) PCT No.: PCT/EP2015/056936

§ 371 Date: Sep. 27, 2016

(30) Foreign Application Priority Data
Apr. 4, 2014 (EP) 14163495.6

(51) Int. Cl. A61M 1/00; B08B 9/023; B08B 9/032; A61L 2/10; F04B 37/14; F04B 23/02; A61L 2/18; A61L 2/24; B08B 7/00; B08B 9/035

(52) U.S. Cl. CPC A61M 1/0066; B08B 7/0057; B08B 9/023; B08B 9/0328; B08B 9/0325; B08B 9/035; F04B 37/14; F04B 23/02; A61L 2/18; A61L 2/24; A61L 2/10; A61M 2209/10; A61L 2202/24; A61L 2202/17

(57) ABSTRACT

A device for cleaning a medical vacuum pump, wherein the medical vacuum pump has a suction connector and an air removal opening, which are connected to each other in a fluid-tight manner. The device has a rinsing agent delivery line, connectable to at least one rinsing agent container and having a first port for connection to the suction connector, and a suction line having a second port for connection to the air removal opening and having a vacuum port for connection to a suction source. A rinsing agent can be sucked from the rinsing agent container through the delivery line and via the first port into the medical vacuum pump and from there via the second port through the suction line into an outflow. This device permits simple and rapid cleaning of a vacuum path of medical vacuum pumps.

### CROSS-REFERENCE TO RELATED APPLICATION

The present application is the US national phase of International Patent Application No. PCT/EP2015/056936, filed Mar. 31, 2015, which application claims priority to European Application No. EP 14163495.6, Apr. 4, 2014. The priority application, EP14163495.6, is hereby incorporated by reference.

### TECHNICAL FIELD

The present invention relates to a device for cleaning a medical vacuum pump.

### PRIOR ART

Reusable portable drainage pumps, e.g. for wound drainage or for thorax drainage, have to be cleaned after use, before they can be used again for the next patient. The housing of the pump is usually wiped and disinfected. The interior of the pump, in particular the internal hoses or the entire vacuum path, cannot be cleaned. It is therefore customary to protect the interior of the pump and the internal hoses by means of a bacteria filter and overflow filter. However, this protection by filters is often inadequate in practice. Small particles or elements can still get into the vacuum path. Moreover, in the event of excessive cleaning of the outside, there is the danger of cleaning liquid getting into the interior of the appliance and making the pump inoperable.

WO 2004/098801 discloses a device for cleaning catheters, in which water is flushed into the catheter via a pressure line and sucked out again via a suction pump. Valves regulate the water pressure.

WO 2011/003419 describes a rinsing device for endoscopes, wherein a cleaning liquid is pumped through the lumen of the endoscope by means of a peristaltic pump.

However, these devices are not designed for cleaning medical drainage pumps.

### DISCLOSURE OF THE INVENTION

It is therefore an object of the invention to create a device for cleaning a medical vacuum pump, in particular a vacuum diaphragm pump, which device also permits cleaning of the internal hoses of the pump.

The device according to the invention is used for cleaning a medical vacuum pump that has a suction connector and an air removal opening, which are connected to each other in the interior of the vacuum pump via a path. The path is preferably fluid-tight. It is preferably the vacuum path of the vacuum pump.

The device according to the invention has
a rinsing agent delivery line, connectable to at least one rinsing agent container and having a first port for connection to the suction connector, and
a suction line having a second port for connection to the air removal opening and having a vacuum port for connection to a suction source.

This device allows a rinsing agent to be sucked from the at least one rinsing agent container through the delivery line and via the first port into the medical vacuum pump and from there via the second port through the suction line into an outflow.

By virtue of this device with the suction source located downstream from the vacuum pump to be cleaned, the internal lumens of a medical suction pump can also be cleaned. The vacuum path of the pump can thus be easily rinsed with a cleaning fluid, in particular a liquid, disinfected and decontaminated. Different kinds of rinsing agent can be sucked through the vacuum path. Rinsing agents are, for example, soap solutions, disinfection solutions, and air. The vacuum path can also be dried finally by flushing with air.

For the cleaning, it is not necessary to open the housing of the pump and expose the pump assembly and the hoses. Instead, the appliance in its entirety, i.e. substantially as it is also used by the patient, can be placed into the cleaning device and cleaned there. No specialist personnel are needed to carry out this cleaning. In particular, the pump does not have to be dismantled and then put together again.

The medical vacuum pump is, for example, a drainage pump, for example for wound drainage or thorax drainage, or a breastpump for expressing human breastmilk. It can be a piston pump, for example, or a diaphragm pump.

Said suction connector of the medical vacuum pump is preferably the suction connector used to apply the vacuum to the patient or to the mother. For cleaning, however, it is also possible for a second suction connector to be provided on the medical vacuum pump, in which case the suction connector used for sucking or expressing is preferably closed before the cleaning.

Said air removal opening is preferably the so-called exhaust of the medical vacuum pump, through which the air displaced in the pump during generation of the underpressure escapes to the outside. Analogously to the suction connector, a second air removal opening can also be specifically present here for the cleaning. In this case, the air removal opening used is preferably also closed during use.

The connection between the cleaning device and the medical vacuum pump to be cleaned is made simpler if the first and second ports of the cleaning device are simple plug connections. In this case, the medical vacuum pump can be easily plugged in. The appliance is switched on manually or automatically, and the cleaning can be carried out automatically. In another embodiment, the pump is placed into the device and the plug connection takes place automatically, by means of the corresponding ports of the device being connected, under the effect of a motor or pneumatically, to the corresponding ports of the pump.

In one embodiment, the suction source is a component part of the device, i.e. a suction pump, preferably a piston pump or a diaphragm pump, is present. In other embodiments, the device has a port for connection to an external suction source, for example to a centralized hospital vacuum system. In the latter case, the device preferably has a regulator valve for regulating the external vacuum.

In a simple embodiment, the medical vacuum pump to be cleaned is connected externally to the device. In a preferred embodiment, the device has a cleaning chamber into which the medical vacuum pump is placed. This chamber can preferably be closed with a lid, and it is preferably able to be closed in a liquid-tight and/or gas-tight manner.

This cleaning chamber is preferably equipped with at least one device for cleaning an outer surface of the medical vacuum pump. This device is, for example, at least one UV-C light source and/or a spraying device and/or a wiping device. In addition, a drying device, for example a fan, can be present for drying the outer surface.

In one embodiment, the at least one rinsing agent container is an external rinsing agent container arranged at a distance from the device. In other embodiments, at least one of the rinsing agent containers is arranged on or in the device according to the invention, in a corresponding seat. In this variant, all of the rinsing agent containers are preferably held on or in the device.

In simple embodiments, the at least one rinsing agent container can be arranged on the outside of the device or it can stand in the cleaning chamber itself. However, the device preferably has an equipment chamber for the at least one rinsing agent container, which equipment chamber is separated from the cleaning chamber by at least one partition wall. In this case, first and second ports are preferably arranged in the partition wall.

In one embodiment, the device has a fluid collection container, wherein the outflow opens into this fluid collection container. In another embodiment, the outflow leads outwards into the wastewater system of the hospital or into an external tank.

In one embodiment, the device comprises a valve unit connected to the delivery line for controlled delivery of rinsing agent from the at least one rinsing agent container and/or of air into the delivery line. If several rinsing agent containers are present, the valve unit makes it possible to switch from a first rinsing agent to a second rinsing agent or to air. It is also possible for rinsing agents to be delivered one after another. Preferably, the valve unit is connected to an electronic control unit, such that an automatic cleaning procedure can be carried out in which various rinsing agents and, if appropriate, air are sucked in a predefined sequence and at predefined quantities and time intervals through the pump that is to be cleaned.

Preferably, the device has a fluid detection sensor connected to the suction line. In one embodiment, the sensor detects whether rinsing agent has been sucked through the whole vacuum path. If not, the device can generate an error message or, depending on the embodiment, can perform an automatic error recognition and, again depending on the embodiment, an error correction.

In a simple embodiment, the pump assembly of the medical pump is inactive during the cleaning, i.e. the pump itself does not provide suction. In another embodiment, however, the pump is switched on and sucks the cleaning fluid through the vacuum channel. This has the advantage that the individual elements of the pump are in motion and, therefore, all areas can be thoroughly cleaned.

The device preferably has cleaning programs in which the suction source and, if appropriate, also the pump to be cleaned are only intermittently in operation. Although the pump to be cleaned then supports the passage of the rinsing agent through the vacuum path, the rinsing agent can nevertheless also remain for some time without moving in the path and exert its action. In a preferred variant, the rinsing agent is sucked into the vacuum path in a first step until the entire vacuum path is wetted. A period in which the rinsing agent acts is left to elapse, without application of underpressure, and then the rinsing agent is sucked out. This procedure can be repeated, or air can be sucked directly through for drying. The cleaning of the outer surface by light, fluids or other means can take place previously, at the same time or subsequently. It preferably takes place at the same time, in order to minimize the time needed for the cleaning.

In a preferred embodiment, the device has a control unit by means of which the medical vacuum pump can be actuated and/or by means of which data stored in the medical vacuum pump can be retrieved and/or by means of which data can be transmitted to the medical vacuum pump. Preferably, the control unit can fix the hours of operation of the medical drainage pump and, if additional manual maintenance is needed, this maintenance can be indicated.

If the pump to be cleaned is controlled by the device, it is preferable that it cannot simply be switched on and off, but instead individual parameters, such as pump frequency and maximum underpressure, can be specifically set.

The embodiments described above can be combined with one another.

### BRIEF DESCRIPTION OF THE DRAWINGS

Preferred embodiments of the invention are described below with reference to the drawings, which serve only for illustration and are not to be interpreted as limiting the invention. In the drawings:

FIG. 1 shows a schematic view of the device according to the invention for cleaning a medical vacuum pump in a first embodiment;

FIG. 2 shows a schematic view of the device according to the invention for cleaning a medical vacuum pump in a second embodiment;

FIG. 3 shows a schematic view of the device according to the invention for cleaning a medical vacuum pump in a third embodiment, and

FIG. 4 shows a schematic view of the device according to the invention for cleaning a medical vacuum pump in a fourth embodiment.

### DESCRIPTION OF PREFERRED EMBODIMENTS

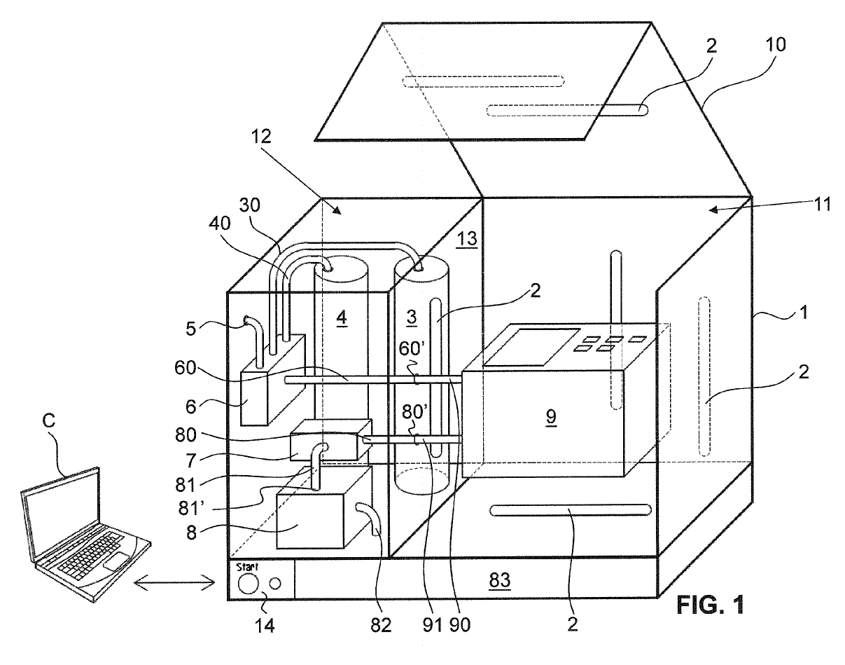

FIG. 1 shows a first illustrative embodiment of a device according to the invention for cleaning a medical vacuum pump 9. The device has a housing 1. In the housing 1, two chambers 11, 12 are separated from each other by a partition wall 13. One of these chambers is a cleaning chamber 11, into which the medical vacuum pump 9 to be cleaned can be placed. Another chamber is designed as an equipment chamber 12.

A pivotable lid 10 permits access to the cleaning chamber 11 and closes same. The equipment chamber 12 is preferably likewise closed. It can likewise have a lid that permits access, although this lid is not shown here.

A fluid collection container 83 is arranged in the floor area of the housing 1 in this example. This fluid collection container 83 is preferably held removably in the housing 1, such that it can be taken out for the purpose of emptying the device.

An electronic control unit 14 is also arranged in the floor of the housing 1, the control panel here being depicted with a start button. An external computer C can preferably communicate with this control unit 14 by customary wireless connection and/or cable connection and can deliver and read data.

The fluid collection container 83 and the control unit 14 can also be arranged in other areas of the housing 1. The same applies to the two chambers 11, 12 and the lid 10. Also in the embodiments described below, the depicted arrangement of the individual elements serves only for illustration and is not to be interpreted as limiting the invention.

At least one rinsing agent container 3, 4 is arranged in the equipment chamber 12. Here, two rinsing agent containers 3, 4 are present. These rinsing agent containers 3, 4 can be designed to be filled by the user or can be supplied to the user as closed disposable cartridges. They contain a rinsing agent, for example a soap solution or a disinfection solution.

From each rinsing agent container 3, 4, a rinsing agent line 30, 40 leads to a valve of a valve unit 6. This valve unit 6 preferably also has an air inlet 5, which is likewise provided with a valve. In this example, the air inlet 5 leads to the environment. However, it is also possible that the air inlet 5 leads to a further container with air, in particular with clean air.

The valve unit 6 is preferably connected to the control unit 14, which opens and closes the individual valves of the unit 6 in a predefined sequence and thus delivers the individual rinsing agents and the air to a delivery line 60. This connection is not shown in FIG. 1.

The delivery line 60 leads through the partition wall 13 into the cleaning chamber 11 and ends in a first port 60′. A first suction line 80 likewise protrudes with a first end through the partition wall 13 and ends there in a second port 80′. With its second end, it leads to a fluid detection sensor 7.

A second suction line 81 leads from the sensor 7 to a suction pump 8. It forms a vacuum port 81′. The first and second suction lines 80, 81 form a common suction line with the second port 80′ and with the vacuum port 81′.

The suction pump 8 is preferably a diaphragm pump. However, it can also be a piston pump or another kind of pump. It can be designed simply as an assembly or it can be enclosed by its own housing in the housing 1.

An outflow line 82 leads from the suction pump into the fluid collection container 83. This container can also be part of the pump 8, such that no further suction line is needed. In particular, the fluid collection container can also be arranged on the vacuum port 81′, and the underpressure needed for sucking the rinsing agent can be generated by the pump 8 in the fluid collection container.

As is shown in FIG. 1, the medical vacuum pump 9 to be cleaned is placed in its entirety, i.e. as it is used by the user, into the cleaning chamber 11. It has a suction connector 90, to which is usually attached the vacuum hose leading through a collection container to the patient or directly to the mother. It also has an air removal opening 91, through which the underpressure generated during use of the pump 9 escapes from the pump assembly. The vacuum path of the medical vacuum pump 9 extends between these two openings 90, 91, said vacuum path including the pump chamber and any other areas needed for generating the vacuum. It is usually fluid-tight.

The suction connector 90 and the air removal opening 91 are shown here schematically. They can be simple openings, for example, or they can be provided with a nozzle. It is also possible that mobile nozzles belonging to the cleaning device are plugged into the ports 90, 91 designed as simple openings.

The ports 90, 91 are connected to the first and second ports 60′, 80′, preferably by a plug connection. The vacuum path of the pump 9 to be cleaned is thereby connected into the cleaning path of the cleaning device. A fluid-tight connection is created from the rinsing agent containers 3, 4, via the valve unit 6 and the delivery line 60 to the suction line 80, 81, the sensor 7 and the suction pump 8 into the fluid collection container 83. The vacuum path of the pump 9 to be cleaned can be rinsed in this way.

The cleaning chamber 11 preferably has a suitable device for cleaning the outside of the medical vacuum pump 9. In this example, this device is a plurality of light sources, preferably UV-C lamps 2, which are distributed on the walls of the cleaning chamber 11, preferably also on the inside of the lid 10, and the pump 9 to be cleaned is thus irradiated as far as possible from all sides. Alternatively or in addition, further devices can also be present, for example a spraying device and/or a wiping device. The position of these devices can be as shown in the figure likewise with the reference number 2.

As has already been discussed in the general part of this description, the control unit 14 can preferably control the pump 9 to be cleaned and switch it on and off during the cleaning procedure and, if appropriate, can also read out data and store new data in the pump 9.

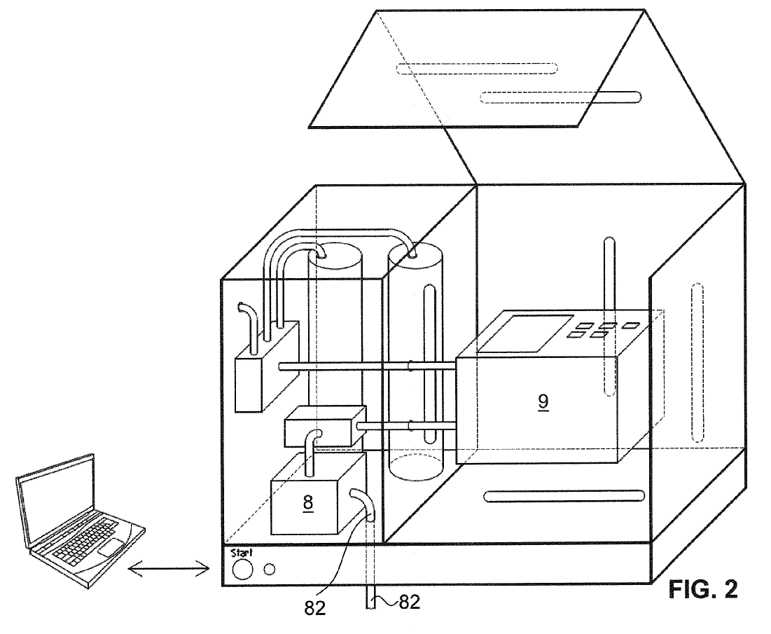

Further embodiments are described below with reference to FIGS. 2 to 4. To facilitate understanding of the figures, not all the reference signs are repeated, and identical or similar parts will be designated in the same way as before and provided with reference signs as in the illustrative embodiment in FIG. 1.

In the example according to FIG. 2, no fluid collection container 83 is present, and instead the outflow line 82 leads from the housing 1 out into an external outflow system or into an external tank.

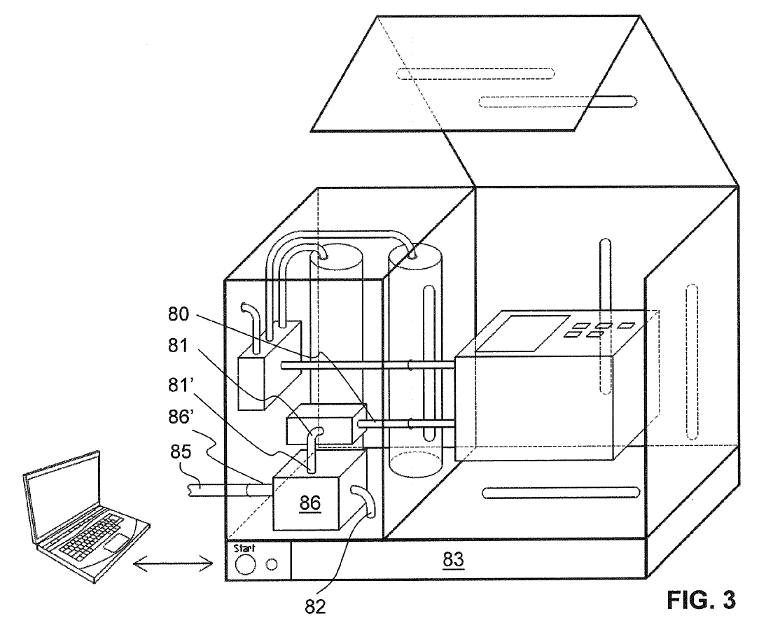

In the example according to FIG. 3, a fluid collection container 83 is once again present. Here, however, the device does not have its own suction pump 8. Instead, a regulator valve 86 is present which can be connected, on the one hand, to the vacuum port 81′ of the suction line 80, 81 and, on the other hand, to an external suction source 85, for example a centralized hospital vacuum system, via a port 86′ for an external suction source. The regulator valve 86 is connected to the control unit 14 and regulates the suction.

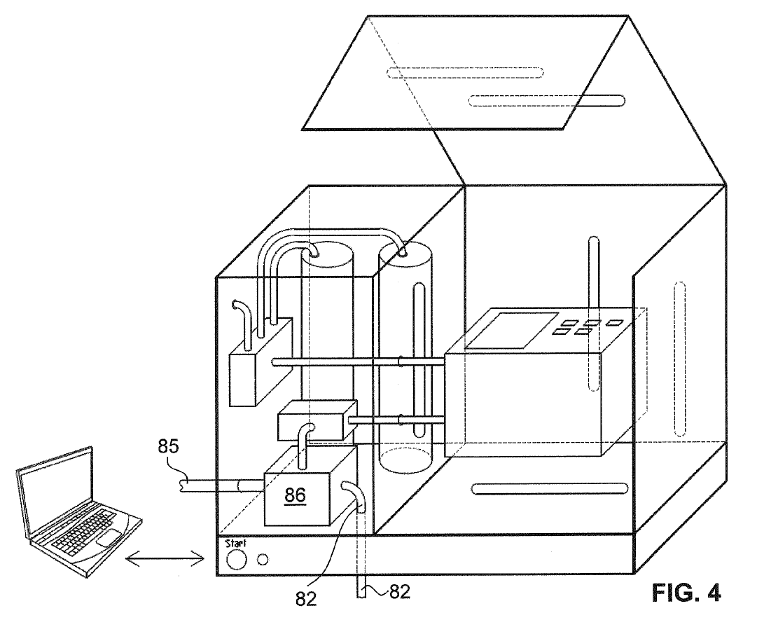

In the example according to FIG. 4, the outflow line 82 leads outwards, and an external vacuum source is used.

The device according to the invention permits simple and rapid cleaning of the vacuum path of medical vacuum pumps.

1. A device for cleaning a medical vacuum pump, wherein the medical vacuum pump has a suction connector and an air removal opening, which are connected to each other in the interior of the vacuum pump via a path, wherein the device has:
a rinsing agent delivery line, connectable to at least one rinsing agent container and having a first port for connection to the suction connector, and
a suction line having a second port for connection to the air removal opening and having a vacuum port for connection to a suction source, wherein a rinsing agent can be sucked from the at least one rinsing agent container through the rinsing agent delivery line and via the first port into the medical vacuum pump and from there via the second port through the suction into an outflow.

2. The device according to claim 1, wherein the first and second ports are plug connections.

3. The device according to claim 1, wherein it furthermore comprises a suction pump, which is connected to the vacuum port of the suction line.

4. The device according to claim 1, wherein it furthermore has a regulator valve, which is connected to the vacuum port and which has a port for connection to an external suction source.

5. The device according claim 1, wherein it has a cleaning chamber for receiving the medical vacuum pump.

6. The device according to claim 5, wherein the cleaning chamber is equipped with at least one device for cleaning an outer surface of the medical vacuum pump.

7. The device according to claim 6, wherein the at least one device for cleaning comprises at least one UV-C light source.

8. The device according to claim 6, wherein the at least one device for cleaning is a spraying device and/or a wiping device.

9. The device according to claim 1, wherein the device has a seat for receiving the at least one rinsing agent container.

10. The device according to claim 5, wherein the device has an equipment chamber, which is separated from the cleaning chamber by at least one partition wall, and wherein the equipment chamber has a seat for receiving the at least one rinsing agent container.

11. The device according to claim 10, wherein the first and second ports are arranged in the partition wall.

12. The device according to claim 1, wherein the device has a fluid collection container, and wherein the outflow opens out in this fluid collection container.

13. The device according to claim 1, wherein the device comprises a valve unit connected to the rinsing agent delivery line for controlled delivery of rinsing agent from the at least one rinsing agent container and/or of air into the rinsing agent delivery line.

14. The device according to claim 1, wherein the device has a fluid detection sensor connected to the suction line.

15. The device according to claim 1, wherein it has a control unit for actuating the medical vacuum pump and/or for retrieving data stored in the medical vacuum pump and/or for transmitting data to the medical vacuum pump.

16. A method for cleaning a medical vacuum pump, comprising:
placing a medical vacuum pump into a cleaning chamber of a cleaning device;
connecting a first port of the cleaning device to a suction connector of the medical vacuum pump via a delivery line;
connecting a second port of the cleaning device to an air removal opening of the medical vacuum pump via a suction line;
switching on the medical vacuum pump;
closing a lid of the cleaning chamber of the cleaning device;
delivering via the delivery line at least one of a rinsing agent from a rinsing agent container, or air, to the suction connector of the medical vacuum pump; and
permitting outflow from the air removal opening to be exhausted, via the suction line, from the medical vacuum pump.

17. The method of claim 16, and in permitting outflow from the air removal opening to be exhausted, via the suction line, from the medical vacuum pump, directing the outflow into a fluid collection container of the cleaning device.

18. The method of claim 16, and in delivering via the delivery line at least one of the rinsing agent from the rinsing agent container, or air, to the suction connector of the medical vacuum pump, operating a valve unit connected to the delivery line to control the delivery.

19. The method of claim 16, and in delivering via the delivery line at least one of the rinsing agent from the rinsing agent container, or air, to the suction connector of the medical vacuum pump, delivering the rinsing agent or air from an equipment chamber of the cleaning device, through a port in a partition separating the cleaning chamber from the equipment chamber.

20. The method of claim 16, further comprising activating one or more external cleaning devices of a group including one or more light sources, a spraying device, and a wiping device.

21. The method of claim 20, and in activating one or more external cleaning devices of the group including one or more light sources, the spraying device, and the wiping device, activating a UV-C lamp.
